(19) United States
(12) Patent Application Publication
(10) Pub. No.: US 2014/0018620 A1
(43) Pub. Date: Jan. 16, 2014

(54) STEERABLE TUBE FOR ENDOSCOPIC APPLICATIONS

(71) Applicant: Marcel Antonius Elisabeth VERBEEK, Nuth (NL)

(72) Inventor: Marcel Antonius Elisabeth VERBEEK, Nuth (NL)

(73) Assignee: FORTIMEDIX B.V., Nuth (NL)

(21) Appl. No.: 13/548,935

(22) Filed: Jul. 13, 2012

(51) Int. Cl. A61M 25/092; A61B 1/01

(52) U.S. Cl. 600/106; 600/149; 604/95.01

(57) ABSTRACT

A steerable tube for endoscopic applications includes a set of longitudinal elements arranged for interconnecting a proximal and a distal end portion of the steerable tube. The set of longitudinal elements is arranged for transferring a movement of the proximal to the distal end portion. The tube further includes an actuator coupled to the set of longitudinal elements at the proximal end portion, for manipulating the set of longitudinal elements, a flexible body that is arranged between the proximal and distal end portions for accommodating the set of longitudinal elements, and a coupling arrangement for establishing an adjustable coupling between the actuator and at least one of the longitudinal elements, thereby allowing the longitudinal elements to assume different lengths between the proximal and distal end portions. An endoscopic instrument, an endoscope and an assembly including a steerable tube set forth are also described.

### FIELD OF THE INVENTION

The present invention relates to a steerable tube for endoscopic applications comprising a set of longitudinal elements that is arranged for interconnecting a proximal end portion and a distal end portion of the steerable tube, wherein the set of longitudinal elements is arranged for transferring a movement of the proximal end portion to the distal end portion. The steerable tube further comprises an actuator coupled to the set of longitudinal elements at the proximal end portion, for manipulating the set of longitudinal elements. Such a steerable tube can be applied in endoscopic equipment, e.g. an endoscopic instrument, an endoscope and an assembly comprising an endoscopic instrument and an endoscope but is not limited thereto.

Equipment for endoscopic applications as described above can be used in medical applications but also in non-medical applications, e.g. inspecting and/or repairing mechanical or electronic installations at locations that are difficult to reach. Therefore, terms used in the following description such as endoscopic application or endoscopic instrument, must be interpreted broadly.

### BACKGROUND OF THE INVENTION

Transformation of surgical interventions that require large incisions for exposing a target area into minimal invasive surgical interventions, i.e. requiring only natural orifices or small incisions for establishing access to the target area, is well-known and ongoing. For performing a minimal invasive surgical intervention, a physician needs to have access to surgical equipment that enables reaching the target area via a small opening, e.g. an incision or natural orifice, and performing actions at the target area in a patient's body. Endoscopic instruments and endoscopes through which these instruments are guided towards a target area are well-known in the art and can comprise a steerable tube as described above. Furthermore these tools may comprise a handle arranged at a proximal end portion of the steerable tube for steering the tube and/or for manipulating a tool that is arranged at a distal end portion of the steerable tube. Such a tool can for example be a camera, a manual manipulator, e.g. a pair of scissors, forceps, or manipulators using an energy source, e.g. an electrical, ultrasonic or optical energy source. In this application, the terms “proximal” and “distal” are defined with respect to an operator, e.g. a physician that operates the endoscopic instrument or endoscope. Thereby is a proximal end portion a portion that is located near the physician and a distal end portion a portion located at a distance of the physician.

Known endoscopic instruments and endoscopes may comprise a steerable tube that is provided with multiple independent movable sections arranged at its respective proximal and distal end portions. Movement of a particular movable section at the proximal end portion in a radial direction of the steerable tube is transferable to a corresponding movable section at the distal end portion via a dedicated set of longitudinal elements. As these sections enable the steerable tubes to assume a broad variety of curvatures, the known endoscopic instruments are provided with an improved steerability of the distal end portion. However, a concern with these known endoscopic instruments is that their construction prevents them to be guided through endoscopes that comprise at least one curve due to a path through which they have been inserted in order to provide access to a target area. It will be clear to the person skilled in the art that a target area that is only accessible via a path comprising a lot of curves such as the gastro-intestinal tract is impossible to access using known endoscopic instruments as described above.

### SUMMARY OF THE INVENTION

It is an object of the present invention to provide a steerable tube for endoscopic applications that preempts or at least reduces the abovementioned concerns of the abovementioned known steerable tubes. It is also an object of the present invention to provide an improved endoscopic instrument comprising a steerable tube according to the invention. It is a further object of the present invention to provide an improved endoscope comprising a steerable tube according to the invention. It is an even further object of the present invention to provide an improved assembly comprising an improved endoscopic instrument and an improved endoscope according to the invention.

At least one of these objects is achieved by a steerable tube according to the present invention comprising a flexible body that is arranged between the proximal and distal end portions of the steerable tube and that is arranged for accommodating the set of longitudinal elements. The steerable tube further comprises a coupling arrangement for establishing an adjustable coupling between the actuator and at least one of the longitudinal elements of the set of longitudinal elements, thereby allowing the longitudinal elements to assume different lengths between the proximal end portion and the distal end portion of the steerable tube. By applying a flexible body, the steerable tube according to the present invention can be guided through an endoscope that follows a path with at least one curve towards a target area. This clearly is an improvement over the known steerable tubes described above. A flexible body is construed as a body having a bending radius typical for known flexible endoscopic equipment. However, depending on a specific application, a flexible body could also be a body comprising otherwise rigid parts that are hingeably connected to each other.

However as the flexible body accommodates the set of longitudinal elements that interconnect the proximal and distal end portions of the steerable tube, any displacement in a radial direction of the flexible body, for example due to bending of the flexible body when it is placed in a tortuous path, results in a radial displacement of at least one of the longitudinal elements. This results in an undesired displacement of the distal end portion in a radial direction of the steerable tube when the proximal end portion is in a preferred position, e.g. a fixed position. Consequently, the orientation of the distal end portion and of a tool that may be connected to it are dependent on a spatial arrangement of the flexible body of the steerable tube that in its turn is determined by the path towards the target area. In this way, the orientation of the distal end portion and/or the tool connected to it is unpredictable. It will be clear to the skilled person that this may impede or prevent guiding of the steerable tube through a lumen of an endoscope. The unpredictable orientation of the distal end portion of the steerable tube may also cause an unexpected orientation of the distal end portion and the tool that may be connected to it when the distal end portion exits the endoscope at the target area. In addition, the tube may have a steering characteristic that is different than a steering characteristic of a steerable tube of which the flexible body is not bent. As a result of the dependency of the orientation of the distal end portion on the bending of the flexible body, the steerable tube may have a deteriorated operability because the operator, e.g. a physician, may be confronted with a different or unexpected response of the distal end portion on a manipulation of the proximal end portion or a limited functionality such as a limited freedom of movement of the tool that is arranged at the distal end of the steerable tube. A different or unexpected response may have a negative effect on the eye-hand coordination of the operator. In addition, body tissue and/or organs could be damaged due to collisions with the tool arranged at the distal end portion of the steerable tube due to an unexpected response and/or orientation of the distal end portion upon leaving an endoscope and entering a target area. Especially in emergency situations in which there is no time for getting adjusted to an unusual steering behavior and/or in situations in which very accurate positioning and maneuvering of the tools is of utmost importance the concerns described above might result in dangerous situations.

In order to remedy the concerns due to the undesired dependency of the orientation of the distal end portion on the bending of the flexible body, the steerable tube is provided with a coupling arrangement for enabling an adjustable coupling between the actuator and at least one of the longitudinal elements of the set of longitudinal elements, thereby allowing the longitudinal elements to assume different lengths between the proximal and the distal end portions of the steerable tube. In this way, a path length difference between the proximal and distal end portions due to bending of the flexible body may be compensated. The different lengths that the longitudinal elements need to assume for compensating the path length difference depend on the spatial arrangement that the flexible body assumes upon being inserted into an endoscope that is arranged in a path comprising a lot of curves, e.g. the gastro-intestinal tract. The coupling arrangement can be arranged for releasing a coupling between the actuator and at least one of the longitudinal elements of the set of longitudinal elements to allow at least one of the longitudinal elements to assume a length that is required for compensating the path length difference between the proximal and distal end portions and thereby rendering the orientation of the distal end portion independent from the bending of the flexible body of the steerable tube. In addition, the distal end portion is not controllable by the actuator due to the released coupling described above. In this case, the distal end portion is enabled to move freely when being guided through a curved trajectory and the orientation of the distal end portion is determined by interaction with at least one boundary of the curved trajectory, e.g. a natural trajectory in a human body like the gastro-intestinal tract or a trajectory in an endoscope.

However, it will be clear to the person skilled in the art that the coupling arrangement can also be arranged for establishing a coupling between the actuator and at least one of the longitudinal elements to fix the length of at least one of the longitudinal elements for rendering the orientation of the distal end portion independent from the bending of the flexible body. After at least one of the longitudinal elements of the set has assumed its length for compensating the path length difference between the proximal and distal end portions, the orientation of the distal end portion is independent of the spatial arrangement of the flexible body, provided that the spatial arrangement of the flexible body substantially remains the same. It will be clear that in case the steerable tube needs to be repositioned, the coupling between the actuator and at least one of the longitudinal elements needs to be adjusted.

Therefore, a steerable tube according to the invention that is applied in an endoscope or in an endoscopic instrument allows them to be placed in a tortuous path while the steerability of the distal end portion remains functional and predictable. It will be clear to the person skilled in the art that this advantage equally applies to an endoscope comprising a steerable tube according to the invention that at its distal end is provided with for example a camera. This capability is a significant improvement over endoscopic instruments and endoscopes known in the art which do not allow this. Furthermore, the steerable tube according to the invention enables the distal end portion of the steerable tube of an endoscopic instrument or of an endoscope to assume any preferred orientation. Moreover, the steerable tube according to the invention provides an endoscopic instrument or an endoscope in which it is applied with a steering characteristic that is identical to the steering characteristic of a steerable tube of which the flexible body is not bent.

In an embodiment of the steerable tube according to the present invention, the adjustable coupling established by the coupling arrangement is one of a friction coupling and a form closed coupling. Such a coupling may be adjusted reliably and can efficiently be established between the actuator and the set of longitudinal elements. A friction coupling may be established by releasably clamping the coupling arrangement and at least one of the longitudinal elements together. A form closed coupling can be established by an engagement of interlocking structures that are arranged on the coupling arrangement and the longitudinal elements.

In an embodiment of the steerable tube according to the present invention, the steerable tube further comprises a controlling arrangement for controlling the coupling arrangement, wherein the controlling arrangement is capable of assuming a first position in which the coupling arrangement is in a state for enabling the at least one longitudinal element of the set of longitudinal elements and the actuator to be moveable with respect to each other, and wherein the controlling arrangement is capable of assuming a second position in which the coupling arrangement is in a state for fixing the at least one longitudinal element of the set of longitudinal elements and the actuator with respect to each other. When the controlling arrangement is in the first position its cooperation with the coupling arrangement is adapted for releasing the coupling between the actuator and at least one of the longitudinal elements of the set of longitudinal elements to allow them to move freely with respect to each other. In this state of the coupling arrangement, the coupling between the actuator and at least one of the longitudinal elements can be adjusted by allowing at least one of the longitudinal elements to assume a length that is required for compensating a path length difference between the proximal and distal end portions and thereby rendering the orientation of the distal end portion of the steerable tube to be independent from the bending of the flexible body. When the controlling arrangement is in the second position its cooperation with the coupling arrangement is adapted for fixing the coupling between the actuator and at least one of the longitudinal elements of the set of longitudinal elements. In this state of the coupling arrangement, at least one of the longitudinal elements of the set of longitudinal elements has assumed a length that in dependence of the path towards the target area is required for compensating the path length difference between the proximal and distal end portions thereby rendering the orientation of the distal end portion independent from the bending of the flexible body of the steerable tube.

In an embodiment of the steerable tube according to the present invention, the controlling arrangement and the coupling arrangement are arranged coaxially. In this way it is possible to adapt the controlling arrangement for engaging the coupling arrangement around its circumference. This may enable an improved control of the controlling arrangement over the coupling arrangement. In addition, if the coupling arrangement and the controlling arrangement are coaxially arranged in or at the steerable tube, the tube may be self-contained with respect to the functionality of enabling compensation of path length differences between the proximal and distal end portions due to bending of the flexible body by adjusting the length of at least one of the longitudinal elements, thereby adjusting the coupling between the actuator and at least one of the longitudinal elements. As a result of this it may be possible to couple any handle known from the art to the steerable tube according to the present invention. As there is no special requirement with respect to the handle for establishing the adjustable coupling, this may provide a cost benefit. The fact that any handle can be coupled to the steerable tube implies that by decoupling the handle and steerable tube after use of an endoscopic instrument and/or endoscope in which the steerable tube is applied allows improved cleaning and sterilizing of the steerable tube. This is beneficial for patient safety upon re-using the endoscopic instrument and/or endoscope.

The coaxial arrangement of the coupling and controlling arrangements may also be advantageous for their manufacture. They may be fabricated from full cylindrical tube-like elements by removing parts of the cylindrical wall by any known material removal technique, such as laser cutting. This is the same technique as is used for the fabrication of the flexible proximal and distal end portions of the steerable tube and for the longitudinal elements. This has been described in detail in international patent applications WO 2009/112060 and WO 2009/127236 of the applicant and in U.S. patent application Ser. No. 13/160,949 of the applicant, which are here incorporated by reference.

It will be clear to the person skilled in the art that it would also be possible to fabricate the coupling and controlling arrangement using injection molding, water jet cutting, etching or electrical discharge machining (edm).

The coaxial arrangement of the coupling and controlling arrangements may also enable a compact implementation of these arrangements.

In an embodiment of the steerable tube according to the present invention, a dimension of the controlling arrangement is variable at a position of the coupling arrangement. The controlling arrangement can be brought into the first and the second position respectively by varying one of its dimensions for example its diameter. As a result of varying the dimension of the controlling arrangement at the position of the coupling arrangement, the latter can be brought in the first and the second state respectively. By adapting the controlling arrangement to have a first diameter, the controlling arrangement is brought in the first position and the coupling arrangement is brought in a first state in which the coupling between the actuator and at least one of the longitudinal elements can be adjusted by allowing at least one of the longitudinal elements to assume a length that is required for compensating a path length difference between the proximal and distal end portions, thereby rendering the orientation of the distal end portion of the steerable tube to be independent from the bending of the flexible body. By adapting the controlling arrangement to have a second diameter, the controlling arrangement is brought in the second position and the coupling arrangement is brought in a second state in which the coupling between the actuator and at least one of the longitudinal elements of the set of longitudinal elements is fixed.

In an embodiment of the steerable tube according to the present invention, the coupling arrangement comprises a protrusion that is arranged for engaging with the at least one longitudinal element of the set of longitudinal elements, wherein the protrusion is adapted to engage with the at least one longitudinal element of the set of longitudinal elements when the controlling arrangement is in the second position. In this way, the adjustable coupling can be implemented as a friction coupling by clamping together the coupling arrangement and at least one of the longitudinal elements via the protrusion. It will be clear to the person skilled in the art that the protrusion can have any suitable shape allowing a compact construction of the steerable tube in the radial direction. It is also possible that upon engaging with the protrusion at least one of the longitudinal elements is brought into contact with another friction element, thereby establishing a clamping coupling.

In an embodiment of the steerable tube according to the present invention, the protrusion comprises at least a first serrated portion, and wherein the at least one longitudinal element comprises at least a second serrated portion for engaging with the first serrated portion of the protrusion. In this way, the adjustable coupling can be implemented as a form closed coupling by an engagement of interlocking structures that are established by the serrated portions on the protrusion and at least one of the longitudinal elements. It will be clear to the person skilled in the art that the first and second serrated portions can also engage with each other via an intermediate element. It will also be clear that the serrated portions have to be implemented for not impeding releasing of the adjustable coupling when the controlling arrangement is brought into the first position.

In an embodiment of the steerable tube according to the present invention, the controlling arrangement comprises a resilient element which comprises two end portions that are arranged for being movable with respect to each other. In this way, the diameter of the resilient element can be varied and consequently the diameter of the controlling arrangement can be changed. The resilient element can be a spring-like element, for example a torsion spring, or a tube-like element that is provided with slits at its circumference as described above, wherein these slits allow the diameter of the tube-like element to be varied. The resilient element can also be a tube-like element comprising for example rubber. In the case of the torsion spring or the tube-like element that is provided with slits at its circumference their diameters can be varied by rotating end parts of the torsion spring and the tube-like element in opposite directions with respect to each other. The diameter of the tube-like element comprising rubber can be varied by translation of its end parts with respect to each other, e.g. by compressing the rubber tube-like element its diameter can be reduced.

In an embodiment of the steerable tube according to the present invention, the controlling arrangement comprises a hinge element having an eccentric shaft. In this way, the controlling arrangement can assume two positions, i.e. a first position in which it allows the coupling arrangement to assume a state in which the coupling between the actuator and at least one of the longitudinal elements can be adjusted by allowing them to move with respect to each other, and a second position in which it allows the coupling arrangement to fix the coupling between the actuator and at least one of the longitudinal elements of the set of longitudinal elements.

In an embodiment of the steerable tube according to the present invention, the controlling arrangement comprises a tapered element that is movable axially along the steerable tube. In this way, the controlling arrangement can also assume the two positions described above. It will be clear to the person skilled in the art that the tapered element can be implemented in many ways, for example as a ring-like element with a conical bore.

In an embodiment of the steerable tube according to the present invention, the steerable tube further comprises a tensioning arrangement for tensioning the set of longitudinal elements. In this way, the lengths of the longitudinal elements of the set of longitudinal elements between the proximal and distal end portions of the steerable tube can be adjusted while keeping the longitudinal elements tensioned. The tensioning arrangement may also be used for positioning the distal end portion in a preferred position and/or restoring a preferred position of the distal end portion upon exiting an endoscope. The tensioning arrangement can comprise a biased spring-like element. It will be clear to the person skilled in the art that the tensioning arrangement can be implemented in many different ways.

In an embodiment of the steerable tube according to the present invention, the controlling arrangement has a displaceable arrangement at the steerable tube. In this way, the steerable tube is self-contained concerning the functionality of adjusting the coupling between the actuator and at least one of the longitudinal elements of the set of longitudinal elements. As a result of this it may be possible to couple any handle known from the art to the steerable tube according to the present invention. As there is no special requirement with respect to the handle for establishing the adjustable coupling, this may provide a cost benefit. The fact that any handle can be coupled to the steerable tube implies that by decoupling the handle and steerable tube after use of an endoscopic instrument and/or endoscope in which the steerable tube is applied allows improved cleaning and sterilizing of the steerable tube. This is beneficial for patient safety upon re-using the endoscopic instrument and/or endoscope.

In an embodiment of the steerable tube according to the present invention, the steerable tube further comprises a handle that is arranged at the proximal end portion for cooperating with the actuator. The handle can be used for steering the distal end portion of the steerable tube and/or for operating a tool arranged at the distal end portion. In a case that the steerable tube comprises the controlling arrangement as described above, the handle can be any handle known in the prior art for constituting an piece of endoscopic equipment, e.g. an endoscopic instrument or an endoscope.

In an embodiment of the steerable tube according to the present invention, the handle is further arranged for cooperating with the controlling arrangement. In this way, only manipulation of the handle may be required for adjusting the coupling between the actuator and at least one of the longitudinal elements. As a result, the operator is provided with improved ease of operation of the steerable tube and/or the piece of endoscopic equipment in which it is applied.

In an embodiment of the steerable tube according to the present invention, the steerable tube further comprises a handle that is arranged at the proximal end portion, wherein the handle is further arranged for accommodating the controlling arrangement. In this way, the handle is required for enabling adjustment of the coupling between the actuator and one of the longitudinal elements. Therefore, a dedicated handle will be required for use with a steerable tube according to this embodiment.

According to another aspect of the present invention, an endoscopic instrument is provided comprising a steerable tube set forth.

In an embodiment of the endoscopic instrument according to the present invention, the adjustable coupling established by the coupling arrangement is one of a friction coupling and a form closed coupling.

In an embodiment of the endoscopic instrument according to the present invention, the steerable tube further comprises a controlling arrangement for controlling the coupling arrangement, wherein the controlling arrangement is capable of assuming a first position in which the coupling arrangement is in a state for enabling the at least one longitudinal element of the set of longitudinal elements and the actuator to be moveable with respect to each other, and wherein the controlling arrangement is capable of assuming a second position in which the coupling arrangement is in a state for fixing the at least one longitudinal element of the set of longitudinal elements and the actuator with respect to each other.

In an embodiment of the endoscopic instrument according to the present invention, the steerable tube further comprises a handle that is arranged at the proximal end portion for cooperating with the actuator.

In an embodiment of the endoscopic instrument according to the present invention, the handle is further arranged for cooperating with the controlling arrangement.

According to another aspect of the present invention, an endoscope is provided comprising a steerable tube set forth.

In an embodiment of the endoscope according to the present invention, the adjustable coupling established by the coupling arrangement is one of a friction coupling and a form closed coupling.

In an embodiment of the endoscope according to the present invention, the steerable tube further comprises a controlling arrangement for controlling the coupling arrangement, wherein the controlling arrangement is capable of assuming a first position in which the coupling arrangement is in a state for enabling the at least one longitudinal element of the set of longitudinal elements and the actuator to be moveable with respect to each other, and wherein the controlling arrangement is capable of assuming a second position in which the coupling arrangement is in a state for fixing the at least one longitudinal element of the set of longitudinal elements and the actuator with respect to each other.

In an embodiment of the endoscope according to the present invention, the steerable tube further comprises a handle that is arranged at the proximal end portion for cooperating with the actuator.

In an embodiment of the endoscope according to the present invention, the handle is further arranged for cooperating with the controlling arrangement.

According to another aspect of the present invention, an assembly is provided comprising an endoscopic instrument and an endoscope comprising a steerable tube set forth.

### BRIEF DESCRIPTION OF THE DRAWINGS

These and other features and effects of the present invention will be explained in more detail below with reference to drawings in which preferred and illustrative embodiments of the invention are shown. The person skilled in the art will realize that other alternatives and equivalent embodiments of the invention can be conceived and reduced to practice without departing from the scope of the present invention. In the drawings,

FIG. 1a shows a schematic representation of a steerable tube according to the present invention in which the coupling between the actuator and the set of longitudinal elements is released.

FIG. 1b shows the schematic representation of the steerable tube as shown in FIG. 1a, in which the coupling between the actuator and the set of longitudinal elements is fixed.

FIG. 1c shows the schematic representation of the steerable tube as shown in FIG. 1b, illustrating its steering characteristic.

FIG. 1d shows a schematic representation of a steerable tube according to the present invention comprising two independently steerable sections arranged at the proximal and distal end portions.

FIG. 1e shows the schematic representation of the steerable tube as shown in FIG. 1d, in an exemplary actuated position.

FIG. 1f shows a schematic representation of a steerable tube according to the present invention comprising longitudinal elements that are arranged in a twisted way between the proximal and distal end portions.

FIG. 1g shows the schematic representation of the steerable tube as shown in FIG. 1f, in an exemplary actuated position.

FIG. 2a shows a schematic side view of an embodiment of a coupling arrangement according to the invention when it is in a state for enabling the length of at least one longitudinal element of the set of longitudinal elements to be adjusted.

FIG. 2b shows a schematic view of the embodiment of the coupling arrangement as shown in FIG. 2a, illustrating a displacement of at least one of the longitudinal elements of the set of longitudinal elements for adjusting its length between the proximal and distal end portions of the steerable tube.

FIG. 2c shows a schematic view of the embodiment of the coupling arrangement as shown in FIG. 2b, when it is in a state for fixing the set of longitudinal elements and the actuator with respect to each other. This is done by establishing an adjustable coupling that is implemented as a form closed coupling. The form closed coupling is formed by an engagement of serrated portions located at protrusions of the coupling arrangement and at the longitudinal elements.

FIG. 3a shows a schematic view of a first exemplary embodiment of a controlling arrangement according to the present invention in a first position in which it has a first diameter.

FIG. 3b shows a schematic view of the embodiment of the controlling arrangement as shown in FIG. 3a, wherein the controlling arrangement is in a second position in which it locally has a second diameter.

FIG. 4a shows a schematic view of a second exemplary embodiment of the controlling arrangement, wherein it is arranged in a first position.

FIG. 4b shows a schematic view of the embodiment of the controlling arrangement as shown in FIG. 4a, wherein it is arranged in a second position.

FIG. 5a shows a schematic view of a third exemplary embodiment of the controlling arrangement, wherein it is arranged in a first position.

FIG. 5b shows a schematic view of the embodiment of the controlling arrangement as shown in FIG. 5a, wherein it is arranged in a second position.

The figures are not necessarily drawn to scale. In the figures identical components are denoted by the same reference numerals.

### DETAILED DESCRIPTION OF EMBODIMENTS

The steerable tube as shown in the annexed drawings can be applied in endoscopic instruments and endoscopes for medical applications but its use is not restricted to that. It may also be used in other applications such as technical applications in which endoscopic instruments and/or endoscopes are for example used for handling and/or viewing parts of machines or installations which are otherwise difficult to reach. The steerable tube according to the present invention as used in this description will implicitly include these applications.

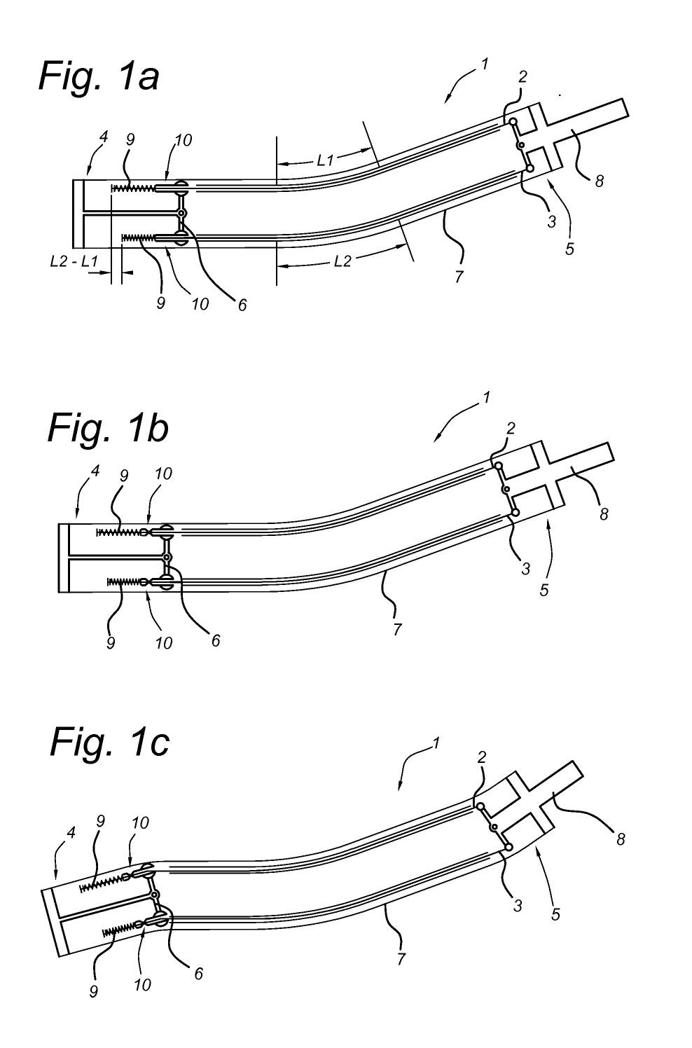

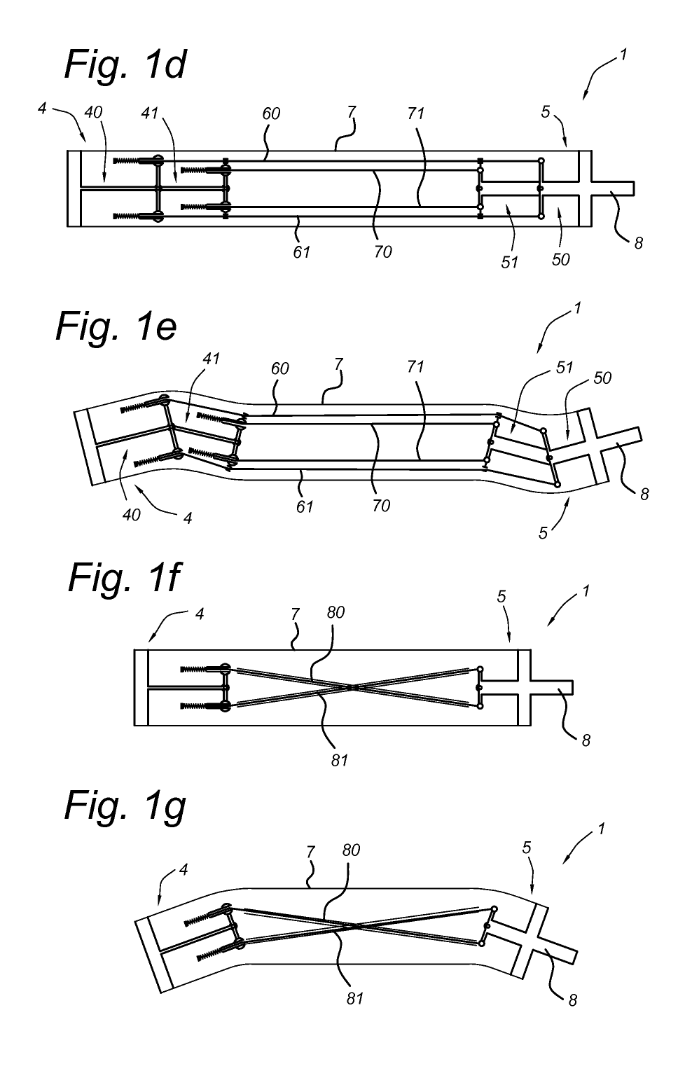

FIG. 1a shows a schematic representation of a steerable tube 1 for endoscopic applications comprising a set of longitudinal elements 2,3 that is arranged for interconnecting a proximal end portion 4 and a distal end portion 5 of the steerable tube 1, wherein the set of longitudinal elements 2,3 is arranged for transferring a movement of the proximal end portion 4 to the distal end portion 5. The steerable tube 1 further comprises an actuator 6 coupled to the set of longitudinal elements 2,3 at the proximal end portion 4, for manipulating the set of longitudinal elements 2,3. FIG. 1a shows that a coupling between the actuator 6 and the set of longitudinal elements 2,3 is released. As a result, the longitudinal elements 2,3 and the actuator 6 can freely move with respect to each other. The steerable tube 1 further comprises a flexible body 7 that is arranged between the proximal 4 and distal 5 end portions of the steerable tube 1. A flexible body is construed as a body having a bending radius typical for known flexible endoscopic equipment. However, depending on a specific application, a flexible body could also be a body comprising otherwise rigid parts that are hingeably connected to each other. It can be seen that the flexible body 7 accommodates the set of longitudinal elements 2,3. In this exemplary embodiment the longitudinal elements 2,3 are represented as cables. However, the longitudinal elements 2,3 may also be elements comprising portions of a wall of a full cylindrical tube, wherein the longitudinal elements 2,3 have been established by providing longitudinal slits in the wall of the full cylindrical tube by any known material removal technique, such as laser cutting or chipping. The fabrication of such longitudinal elements has been described in detail in international patent application WO 2009/112060 of the applicant, which is here incorporated by reference. The steerable tube 1 further comprises a tool 8 that is arranged at the distal end portion 5 of the steerable tube 1 and is connected to the longitudinal elements 2,3 as described above. The tool, which is schematically represented in the appended drawings, can for example be a camera, a manual manipulator, e.g. a pair of scissors, forceps, or manipulators using an energy source, e.g. an electrical, ultrasonic or optical energy source.

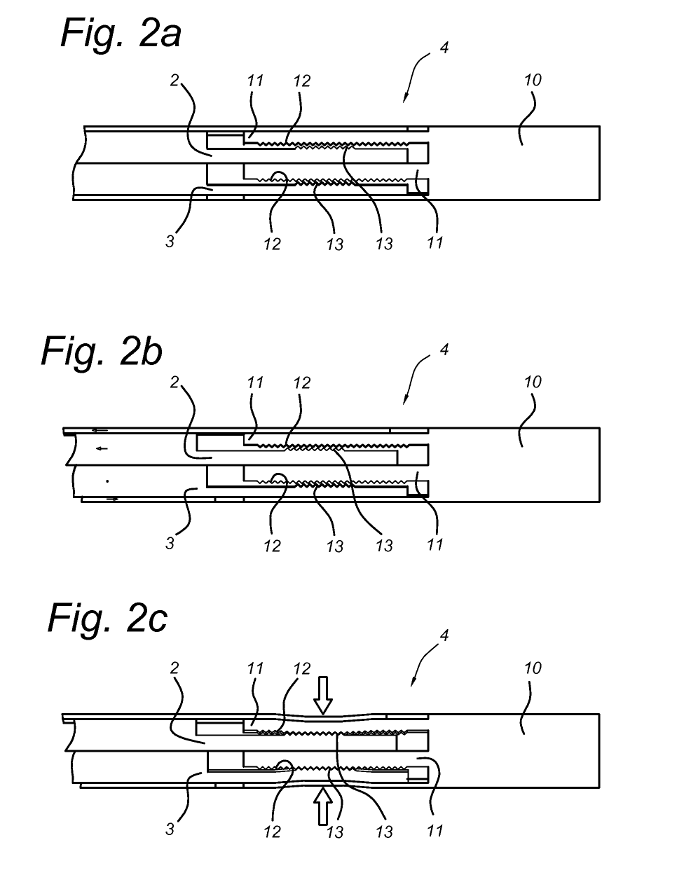

The steerable tube 1 further comprises a coupling arrangement 10, which is shown in more detail in FIGS. 2-5, for establishing an adjustable coupling between the actuator 6 and at least one of the longitudinal elements 2,3 of the set of longitudinal elements, thereby allowing at least one of the longitudinal elements 2,3 to assume a length between the proximal end portion 4 and the distal end portion 5 of the steerable tube 1 required for compensating a path length difference L2-L1 due to bending of the flexible body 7 that accommodates the set of longitudinal elements 2,3. As a result, an undesired dependency of the orientation of the distal end portion 5 on the bending of the flexible body 7 may be remedied. The coupling arrangement 10 can be arranged for releasing a coupling between the actuator 6 and at least one of the longitudinal elements 2,3 of the set of longitudinal elements as is schematically shown in FIG. 1a. In this case, the distal end portion 5 is not controllable by the actuator 6 due to the released coupling described above. However, it will be clear to the person skilled in the art that the coupling arrangement 10 can also be arranged for establishing a coupling between the actuator 6 and at least one of the longitudinal elements 2,3 to fix the length of at least one of the longitudinal elements 2,3 for rendering the orientation of the distal end portion 5 independent from the bending of the flexible body 7. FIG. 1a shows a preferred orientation of the distal end portion 5 when it for example exits an endoscope through which it was guided towards a target area. The steerable tube 1 further comprises a tensioning arrangement 9 for tensioning the set of longitudinal elements 2,3. In this way, the lengths of the longitudinal elements 2,3 of the set of longitudinal elements between the proximal 4 and distal 5 end portions of the steerable tube 1 can be adjusted while keeping the longitudinal elements 2,3 tensioned. In this exemplary embodiment the tensioning arrangement 9 is implemented by biased spring elements. It will be clear to the person skilled in the art that the tensioning arrangement 9 can be implemented in many different ways.

FIG. 1b shows the schematic representation of the steerable tube 1 as shown in FIG. 1a, in which the coupling arrangement 10 is in a state in which the coupling between the actuator 6 and the set of longitudinal elements 2,3 is fixed. In this case the distal end portion 5 is controllable by the proximal end portion 4. This is illustrated in FIG. 1c which shows that a movement of the proximal end portion 4 in a first radial direction of the steerable tube 1 results in a movement of the distal end portion 5 in a second radial direction of the steerable tube 1. The steering characteristic of the exemplary embodiment of the steerable tube 1 shown in FIG. 1c is thus that a movement of the proximal end portion 4 in a first radial direction of the steerable tube results in a movement of the distal end portion 5 in a second radial direction, wherein the first and second radial directions are opposite with respect to each other.

FIG. 1d shows a schematic representation of a steerable tube 1 according to the present invention comprising two independently steerable sections 40,41,50,51 arranged at the proximal 4 and distal 5 end portions. Each of the steerable sections 40, 41 at the proximal end portion 4 is interconnected with a corresponding steerable section 50,51 at the distal end portion 5 by a dedicated set of longitudinal elements 60,61,70,71. Such a steerable tube 1 enables the formation of a broad variety of curvatures, for example S-like curves, as described above and in detail in international application WO 2009/127236 of the applicant, which is here incorporated by reference.

It will be clear to the person skilled in the art that each pair of steerable sections has dedicated coupling 10 and controlling 14 arrangements for adjusting the lengths of the corresponding dedicated sets of longitudinal elements for compensating path length differences between the proximal 4 and distal 5 end portions of the steerable tube 1. The dedicated coupling 10 and controlling 14 arrangements may be arranged for being operable using a handle that is connected to the proximal end portion 4.

FIG. 1e shows the schematic representation of the steerable tube as shown in FIG. 1d, in an exemplary actuated position.

FIG. 1f shows a schematic representation of a steerable tube 1 according to the present invention comprising longitudinal elements 80,81 that are arranged in a twisted way between the proximal 4 and distal 5 end portions. It will be clear to the person skilled in the art that the steerable tube 1 according to the invention can also be provided with longitudinal elements that are arranged in a twistable way between the proximal and distal end portions. Longitudinal elements that are arranged in a twisted arrangement or a twistable arrangement between the proximal and distal end portions are described in detail in U.S. patent application Ser. No. 13/160,949 of the applicant, which is here incorporated by reference.

FIG. 1g shows the schematic representation of the steerable tube as shown in FIG. 1f, in an exemplary actuated position.

FIG. 2a shows a detailed schematic side view of an embodiment of the coupling arrangement 10 according to the invention that is arranged at the proximal end portion 4 of the steerable tube 1 that is only partially shown. The coupling arrangement 10 as shown in FIG. 2a is in a state for enabling the length of at least one longitudinal element 2 of the set of longitudinal elements 2,3 to be adjusted. In this exemplary embodiment the coupling arrangement 10 comprises longitudinal protrusions 11 that are interdigitatedly arranged with the longitudinal elements 2,3. Both the protrusions 11 and the longitudinal elements 2,3 comprise serrated portions 12,13 having interlocking structures, in this example triangles, for establishing a form closed coupling upon engagement of the longitudinal elements 2,3 and the longitudinal protrusions 11. It will be clear to the person skilled in the art that the serrated portions 12,13 can be provided with any suitable structures for establishing a form closed coupling. In the state of the coupling arrangement 10 shown in FIG. 2a, the longitudinal elements 2,3 and the longitudinal protrusions 11 are arranged for allowing them to move freely with respect to each other.

FIG. 2b shows a schematic view of the embodiment of the coupling arrangement 10 as shown in FIG. 2a, illustrating a displacement of longitudinal element 2 for adjusting its length between the proximal 4 and distal 5 end portions of the steerable tube 1 for compensating a path length difference at least between longitudinal elements 2,3 as a result of bending of the flexible body of the steerable tube. In this way, it may be prevented that the distal end portion 5 is displaced due to bending of the flexible body of the steerable tube.

FIG. 2c shows a schematic view of the embodiment of the coupling arrangement 10 as shown in FIG. 2b, when it is in a state for fixing the set of longitudinal elements 2,3 and the actuator to each other. This is done by establishing an adjustable coupling that in this exemplary embodiment is implemented as a form closed coupling. It will be clear to the person skilled in the art that a friction coupling could also be used. The form closed coupling is established by an engagement of the serrated portions 12,13 that are arranged at the longitudinal protrusions 11 and at the longitudinal elements 2,3 respectively. In this exemplary embodiment, the serrated portions 12,13 comprise interlocking triangular structures but it will be clear to the person skilled in the art that other suitable interlocking structures can be used. FIG. 2c illustrates that the engagement between the longitudinal protrusions 11 of the coupling arrangement 10 and the longitudinal elements 2,3 is established by reducing the diameter of the coupling arrangement 10 at a location, indicated by two arrows, at which the longitudinal elements 2,3 and the longitudinal protrusions 11 are interdigitatedly arranged. The diameter of the coupling arrangement 10 can be reduced by applying a force to the coupling arrangement 10 in a radial direction of the coupling arrangement 10. By establishing the coupling between the longitudinal elements 2,3 and the protrusions 11 of the coupling arrangement 10, the actuator and the set of longitudinal elements are coupled. Consequently, the distal end portion 5 can be steered by manipulating the proximal end portion 4 of the steerable tube 1.

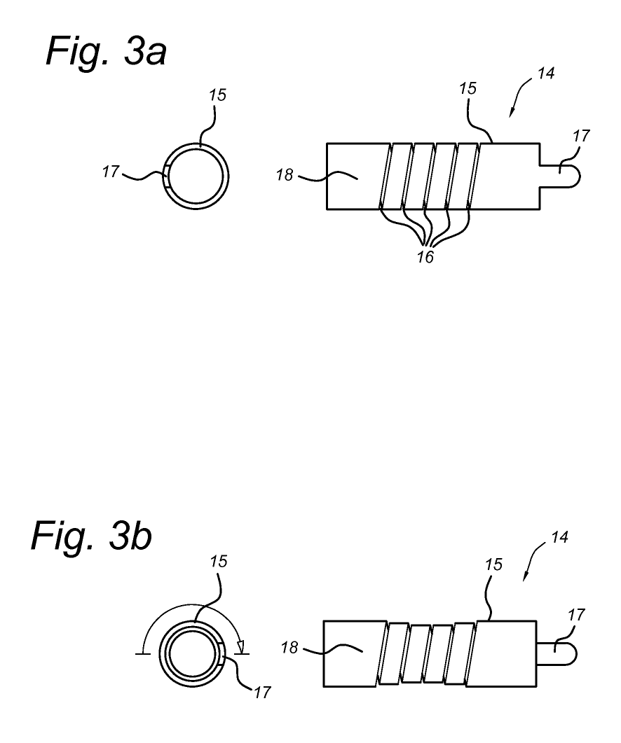

FIG. 3a shows a schematic view of a first exemplary embodiment of a controlling arrangement 14 according to the present invention, wherein the controlling arrangement 14 is a resilient element, in this case a tube-like element 15 that is provided with slits 16 at its circumference. These slits 16 allow the diameter of the tube-like element 15 to be varied by rotating a first end portion 17 and a second end portion 18 of the tube-like element 15 with respect to each other. The controlling arrangement 14 according to this exemplary embodiment and the coupling arrangement 10 are preferably coaxially arranged. The first end portion 17 is for example arranged for enabling manipulation by an operator of the endoscopic instrument or the endoscope in which the steerable tube according to the invention is applied. The second end portion 18 is for example connected to the coupling arrangement 10 that is enclosed by the controlling arrangement 14.

The tube-like element 15 shown in FIG. 3a is in a first position in which it has a first diameter. When the tube-like element 15 is in this first position and is in an coaxial arrangement with the coupling arrangement 10, the coupling arrangement is in a first state in which the coupling between the actuator 6 and at least one of the longitudinal elements 2,3 can be adjusted by allowing at least one of the longitudinal elements 2,3 to assume a length that is required for compensating a path length difference between the proximal and distal end portions. Thereby the orientation of the distal end portion 5 of the steerable tube 1 is independent from the bending of the flexible body 7.

FIG. 3b shows a schematic view of the embodiment of the controlling arrangement 14 as shown in FIG. 3a, wherein the tube-like element 15 is in a second position. In this second position the tube-like element 15 according to this exemplary embodiment locally has a second diameter that is smaller than the first diameter which it has when it is in the first position and which it may still locally have when it is in the second position. The latter situation is illustrated in FIG. 3b. By rotating the first 17 and second 18 end portions of the tube-like element 15 with respect to each other as described above, the tube-like element 15 is brought in the second position and is locally provided with a second diameter. When the tube-like element 15 and the coupling arrangement 10 are coaxially arranged and when the tube-like element 15 is brought in the second position as described above, the coupling arrangement 10 is brought in a second state in which the coupling between the actuator 6 and at least one of the longitudinal elements 2,3 is fixed.

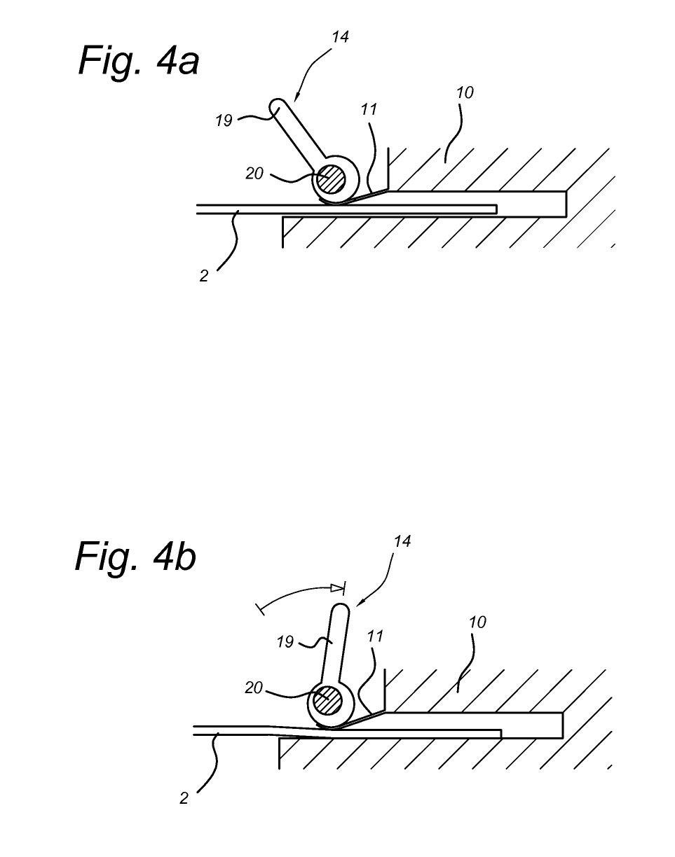

FIG. 4a shows a schematic view of a second exemplary embodiment of the controlling arrangement 14. In this case, the controlling arrangement 14 is a hinge element 19 having an eccentric shaft 20. FIG. 4a shows the hinge element 19 in a first position in which it allows the coupling arrangement 10 to assume a state in which the coupling between the actuator 6 and at least one of the longitudinal elements can be adjusted by allowing them to move with respect to each other.

FIG. 4b shows a schematic view of the embodiment of the controlling arrangement 14 as shown in FIG. 4a, wherein the hinge element 19 is in a second position in which it allows the coupling arrangement 10 to fix the coupling between the actuator 6 and at least one of the longitudinal elements of the set of longitudinal elements by establishing a friction coupling between a protrusion 11 of the coupling arrangement 10 and a longitudinal element 2. It will be clear to the person skilled in the art that a form closed coupling can also be used.

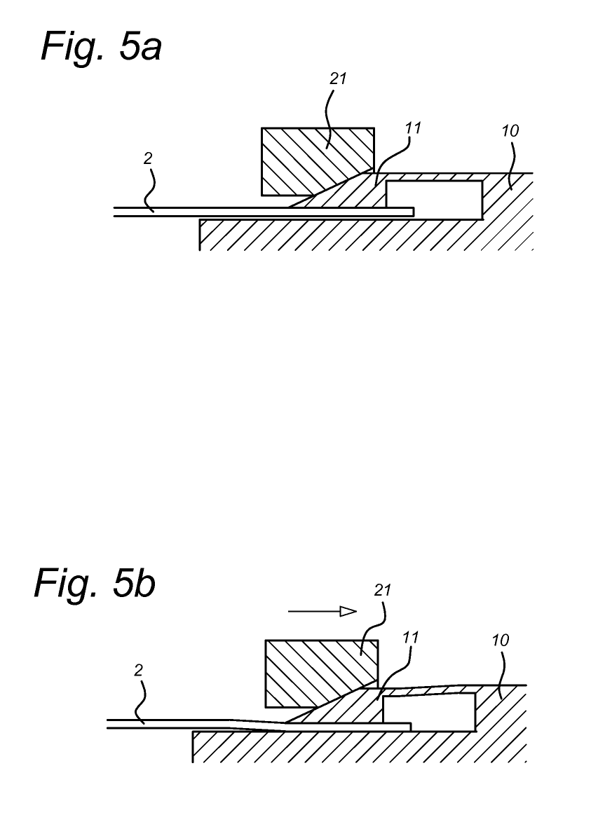

FIG. 5a shows a schematic view of a third exemplary embodiment of the controlling arrangement 14 that is a tapered element 21 that is movable axially along the steerable tube 1, wherein the tapered element 21 is in a first position in which it allows the coupling arrangement 10 to assume a state in which the coupling between the actuator 6 and at least one of the longitudinal elements 2 can be adjusted by allowing them to move with respect to each other.

FIG. 5b shows a schematic view of the embodiment of the controlling arrangement 14 as shown in FIG. 5a, wherein the tapered element 21 is in a second position in which it allows the coupling arrangement 10 to fix the coupling between the actuator 6 and at least one of the longitudinal elements of the set of longitudinal elements by establishing a friction coupling between a protrusion 11 of the coupling arrangement 10 and a longitudinal element 2. It will be clear to the person skilled in the art that a form closed coupling can also be used. It will also be clear to the person skilled in the art that the tapered element 21 can be implemented in many ways, for example as a ring-like element with a conical bore.

The present invention can be summarized by the following clauses:

Clause 1. Steerable tube (1) for endoscopic applications comprising

a set of longitudinal elements (2,3;60,61;70,71;80,81) that is arranged for interconnecting a proximal end portion (4) and a distal end portion (5) of the steerable tube (1), wherein the set of longitudinal elements (2,3;60,61;70,71;80,81) is arranged for transferring a movement of the proximal end portion (4) to the distal end portion (5);

an actuator (6) coupled to the set of longitudinal elements (2,3;60,61;70,71;80,81) at the proximal end portion (4), for manipulating the set of longitudinal elements (2,3;60,61;70,71;80,81);

a flexible body (7) that is arranged between the proximal (4) and distal (5) end portions of the steerable tube (1) and is arranged for accommodating the set of longitudinal elements (2,3;60,61;70,71;80,81);

a coupling arrangement (10) for establishing an adjustable coupling between the actuator (6) and at least one of the longitudinal elements of the set of longitudinal elements (2,3;60,61;70,71;80,81), thereby allowing the longitudinal elements to assume different lengths between the proximal end portion (4) and the distal end portion (5) of the steerable tube (1).

Clause 2. Steerable tube (1) according to clause 1, wherein the adjustable coupling established by the coupling arrangement (10) is one of a friction coupling and a form closed coupling.

Clause 3. Steerable tube (1) according to clause 1 or 2, wherein the steerable tube (1) further comprises a controlling arrangement (14) for controlling the coupling arrangement (10), wherein the controlling arrangement (14) is capable of assuming a first position in which the coupling arrangement (10) is in a state for enabling the at least one longitudinal element of the set of longitudinal elements (2,3;60,61;70,71;80,81) and the actuator (6) to be moveable with respect to each other, and wherein the controlling arrangement (14) is capable of assuming a second position in which the coupling arrangement (10) is in a state for fixing the at least one longitudinal element of the set of longitudinal elements (2,3;60,61;70,71;80,81) and the actuator (6) with respect to each other.

Clause 4. Steerable tube (1) according to clause 3, wherein the controlling arrangement (14) and the coupling arrangement (10) are arranged coaxially.

Clause 5. Steerable tube (1) according to clause 3 or 4, wherein a dimension of the controlling arrangement (14) is variable at a position of the coupling arrangement (10).

Clause 6. Steerable tube (1) according to any of the clauses 3-5, wherein the coupling arrangement (10) comprises a protrusion (11) that is arranged for engaging with the at least one longitudinal element of the set of longitudinal elements (2,3;60,61;70,71;80,81), wherein the protrusion (11) is adapted to engage with the at least one longitudinal element of the set of longitudinal elements when the controlling arrangement (14) is in the second position.

Clause 7. Steerable tube (1) according to clause 6, wherein the protrusion (11) comprises at least a first serrated portion (12), and wherein the at least one longitudinal element comprises at least a second serrated portion (13) for engaging with the first serrated portion (12) of the protrusion (11).

Clause 8. Steerable tube (1) according to any of the clauses 3-7, wherein the controlling arrangement (14) comprises a resilient element (15) which comprises two end portions (17,18) that are arranged for being movable with respect to each other.

Clause 9. Steerable tube (1) according to clause 3, wherein the controlling arrangement (14) comprises a hinge element (19) having an eccentric shaft (20).

Clause 10. Steerable tube (1) according to any of the clauses 3-7, wherein the controlling arrangement (14) comprises a tapered element (21) that is movable axially along the steerable tube (1).

Clause 11. Steerable tube (1) according to any of the preceding clauses, wherein the steerable tube (1) further comprises a tensioning arrangement (9) for tensioning the set of longitudinal elements (2,3;60,61;70,71;80,81).

Clause 12. Steerable tube (1) according to any of the clauses 3-11, wherein the controlling arrangement (14) has a displaceable arrangement at the steerable tube (1).

Clause 13. Steerable tube (1) according to clause 12, wherein the steerable tube (1) further comprises a handle that is arranged at the proximal end portion (4) for cooperating with the actuator (6).

Clause 14. Steerable tube (1) according to clause 13, wherein the handle is further arranged for cooperating with the controlling arrangement (14).

Clause 15. Steerable tube (1) according to any of the clauses 3-11, wherein the steerable tube (1) further comprises a handle that is arranged at the proximal end portion (4), wherein the handle is further arranged for accommodating the controlling arrangement (14).

Clause 16. Endoscopic instrument comprising a steerable tube (1) according to any of the preceding clauses.

Clause 17. Endoscope comprising a steerable tube (1) according to any of the clauses 1-15.

Clause 18. Assembly comprising an endoscopic instrument according to clause 16 and an endoscope according to clause 17.

It will be clear to a person skilled in the art that the scope of the present invention is not limited to the examples discussed in the foregoing, but that several amendments and modifications thereof are possible without deviating from the scope of the present invention as defined in the attached claims. While the present invention has been illustrated and described in detail in the figures and the description, such illustration and description are to be considered illustrative or exemplary only, and not restrictive. The present invention is not limited to the disclosed embodiments but comprises any combination of the disclosed embodiments that can come to an advantage.

Variations to the disclosed embodiments can be understood and effected by a person skilled in the art in practicing the claimed invention, from a study of the figures, the description and the attached claims. In the claims, the word “comprising” does not exclude other steps or elements, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope of the present invention.

What is claimed is:

1. Steerable tube for endoscopic applications comprising
a set of longitudinal elements that is arranged for interconnecting a proximal end portion and a distal end portion of the steerable tube, wherein the set of longitudinal elements is arranged for transferring a movement of the proximal end portion to the distal end portion;
an actuator coupled to the set of longitudinal elements at the proximal end portion, for manipulating the set of longitudinal elements;
a flexible body that is arranged between the proximal and distal end portions of the steerable tube and is arranged for accommodating the set of longitudinal elements;
a coupling arrangement for establishing an adjustable coupling between the actuator and at least one of the longitudinal elements of the set of longitudinal elements, thereby allowing the longitudinal elements to assume different lengths between the proximal end portion and the distal end portion of the steerable tube.

2. Steerable tube according to claim 1, wherein the adjustable coupling established by the coupling arrangement is one of a friction coupling and a form closed coupling.

3. Steerable tube according to claim 1, wherein the steerable tube further comprises a controlling arrangement for controlling the coupling arrangement, wherein the controlling arrangement is capable of assuming a first position in which the coupling arrangement is in a state for enabling the at least one longitudinal element of the set of longitudinal elements and the actuator to be moveable with respect to each other, and wherein the controlling arrangement is capable of assuming a second position in which the coupling arrangement is in a state for fixing the at least one longitudinal element of the set of longitudinal elements and the actuator with respect to each other.

4. Steerable tube according to claim 2, wherein the steerable tube further comprises a controlling arrangement for controlling the coupling arrangement, wherein the controlling arrangement is capable of assuming a first position in which the coupling arrangement is in a state for enabling the at least one longitudinal element of the set of longitudinal elements and the actuator to be moveable with respect to each other, and wherein the controlling arrangement is capable of assuming a second position in which the coupling arrangement is in a state for fixing the at least one longitudinal element of the set of longitudinal elements and the actuator with respect to each other.

5. Steerable tube according to claim 3, wherein the controlling arrangement and the coupling arrangement are arranged coaxially.

6. Steerable tube according to claim 3, wherein a dimension of the controlling arrangement is variable at a position of the coupling arrangement.

7. Steerable tube according to claim 3, wherein the coupling arrangement comprises a protrusion that is arranged for engaging with the at least one longitudinal element of the set of longitudinal elements, wherein the protrusion is adapted to engage with the at least one longitudinal element of the set of longitudinal elements when the controlling arrangement is in the second position.

8. Steerable tube according to claim 7, wherein the protrusion comprises at least a first serrated portion, and wherein the at least one longitudinal element comprises at least a second serrated portion for engaging with the first serrated portion of the protrusion.

9. Steerable tube according to claim 3, wherein the controlling arrangement comprises a resilient element which comprises two end portions that are arranged for being movable with respect to each other.

10. Steerable tube according to claim 3, wherein the controlling arrangement comprises a hinge element having an eccentric shaft.

11. Steerable tube according to claim 3, wherein the controlling arrangement comprises a tapered element that is movable axially along the steerable tube.

12. Steerable tube according to claim 1, wherein the steerable tube further comprises a tensioning arrangement for tensioning the set of longitudinal elements.

13. Steerable tube according to claim 3, wherein the controlling arrangement has a displaceable arrangement at the steerable tube.

14. Steerable tube according to claim 13, wherein the steerable tube further comprises a handle that is arranged at the proximal end portion for cooperating with the actuator.

15. Steerable tube according to claim 14, wherein the handle is further arranged for cooperating with the controlling arrangement.

16. Steerable tube according to claim 3, wherein the steerable tube further comprises a handle that is arranged at the proximal end portion, wherein the handle is further arranged for accommodating the controlling arrangement.

17. Endoscopic instrument comprising a steerable tube according to claim 1.

18. Endoscopic instrument according to claim 17, wherein the adjustable coupling established by the coupling arrangement is one of a friction coupling and a form closed coupling.

19. Endoscopic instrument according to claim 17, wherein the steerable tube further comprises a controlling arrangement for controlling the coupling arrangement, wherein the controlling arrangement is capable of assuming a first position in which the coupling arrangement is in a state for enabling the at least one longitudinal element of the set of longitudinal elements and the actuator to be moveable with respect to each other, and wherein the controlling arrangement is capable of assuming a second position in which the coupling arrangement is in a state for fixing the at least one longitudinal element of the set of longitudinal elements and the actuator with respect to each other.

20. Endoscopic instrument according to claim 17, wherein the steerable tube further comprises a handle that is arranged at the proximal end portion for cooperating with the actuator.

21. Endoscopic instrument according to claim 19, wherein the handle is further arranged for cooperating with the controlling arrangement.

22. Endoscope comprising a steerable tube according to claim 1.

23. Endoscope according to claim 22, wherein the adjustable coupling established by the coupling arrangement is one of a friction coupling and a form closed coupling.

24. Endoscope according to claim 22, wherein the steerable tube further comprises a controlling arrangement for controlling the coupling arrangement, wherein the controlling arrangement is capable of assuming a first position in which the coupling arrangement is in a state for enabling the at least one longitudinal element of the set of longitudinal elements and the actuator to be moveable with respect to each other, and wherein the controlling arrangement is capable of assuming a second position in which the coupling arrangement is in a state for fixing the at least one longitudinal element of the set of longitudinal elements and the actuator with respect to each other.

25. Endoscope according to claim 22, wherein the steerable tube further comprises a handle that is arranged at the proximal end portion for cooperating with the actuator.

26. Endoscope according to claim 24, wherein the handle is further arranged for cooperating with the controlling arrangement.

27. Assembly comprising
an endoscopic instrument that comprises a steerable tube comprising a set of longitudinal elements that is arranged for interconnecting a proximal end portion and a distal end portion of the steerable tube, wherein the set of longitudinal elements is arranged for transferring a movement of the proximal end portion to the distal end portion; an actuator coupled to the set of longitudinal elements at the proximal end portion, for manipulating the set of longitudinal elements; a flexible body that is arranged between the proximal and distal end portions of the steerable tube and is arranged for accommodating the set of longitudinal elements; a coupling arrangement for establishing an adjustable coupling between the actuator and at least one of the longitudinal elements of the set of longitudinal elements, thereby allowing the longitudinal elements to assume different lengths between the proximal end portion and the distal end portion of the steerable tube; and
an endoscope according to claim 22.
